(19) United States
(12) Patent Application Publication
(10) Pub. No.: US 2026/0232476 A1
(43) Pub. Date: Aug. 13, 2026

(54) Internal Nasal Dilator

(71) Applicant: Hushi WANG, Beijing (CN)

(72) Inventor: Hushi WANG, Beijing (CN)

(21) Appl. No.: 19/160,673

(22) PCT Filed: Jul. 21, 2023
(86) PCT No.: PCT/CN2023/108548

§ 371 Date: Aug. 28, 2025

(51) Int. Cl. A61F 5/08

(52) U.S. Cl. CPC A61F 5/08

(57) ABSTRACT

The present invention relates to the technical field of medical instruments, and relates to an internal nasal dilator. The device comprises: a nose clip and two annular intranasal stents symmetrically arranged in the two nasal passages. The nose clip half surrounds and is secured at the lower end of the nasal septum; the two intranasal stents are detachably connected and fixed to two branches of the nose clip, respectively; and a user adjusts the size of the internal nasal dilator by adjusting the perimeter of each annular intranasal stent. The present invention compactly, safely, and reliably combines the three advantageous technologies of separable structures, annular intranasal stents and size adjustment, and comprehensively improves the performance of internal nasal dilators.

### TECHNICAL FIELD

The present invention relates to the technical field of medical instruments, and specifically to an internal nasal dilator.

### BACKGROUND

A significant number of people suffer from nasal obstruction. Nasal obstruction predominantly occurs in the nasal valve region, which is the narrowest segment of the nasal passage. This region is located deep within the nasal vestibule, and represents the position of the highest respiratory resistance in the nasal passage. Nasal obstruction may be triggered once tissues in the nasal valve region undergo swelling, hyperplasia, or hypertrophy or deviation of the nasal septum in this region. Nasal obstruction can lead to symptoms such as impaired nasal breathing, mouth breathing, and snoring, causing inconvenience in patients' daily lives.

An internal nasal dilator is a medical instrument that alleviates nasal obstruction through physical means, of which the core structure includes two intranasal stents arranged in nasal passages on two sides and a central portion that is located near the nasal septum and is used for connecting the two intranasal stents. The intranasal stents support and dilate the nasal passages from the inner sides, especially dilate nasal valve regions, thereby enhancing airflow in the nasal passages.

Existing internal nasal dilators may be categorized into two types based on sources of dilating forces. The first type is an “external nasal elastic dilator”, which uses a resilient force of a central portion located outside the nose to dilate nasal passages and includes intranasal stents used for transmitting the resilient force to a nasal ala inner wall. The invention patent entitled Nasal device for improving nasal breathing (Application Publication No.: US20210085509A1), the invention patent entitled NASAL DILATOR AND USE THEREOF (Application Publication No.: JP2012016602A), the invention patent entitled Nasal dilator with columella retainer for resisting withdrawal and adjustable sections for optimizing fit, comfort, and breathing performance (Application Publication No.: U.S. Pat. No. 11,628,083B1), and the invention patent entitled Adjustable nasal dilator filter (Application Publication No.: U.S. Pat. No. 6,863,066B2) are all internal nasal dilators of this type. This type of internal nasal dilator has the advantages of a simple structure, a strong localized support force for the nasal alae, and good versatility, but has disadvantages as follows: First, because the central portion that provides the resilient force is far away from the nasal alae and the force direction of the dilating force on the nasal ala inner wall is unfavorable for the fixation of the internal nasal dilator, the internal nasal dilator is prone to torsional deformation, and it is difficult to wear it stably. Second, the dilating force of the internal nasal dilator concentrates on the nasal ala position, resulting in high localized pressure that may easily irritate the nasal cavity and cause discomfort. Third, the body of the internal nasal dilator spans the central portion of the nasal passages on two sides, creating a significant resistance during respiration.

The second type of internal nasal dilator is an “internal nasal elastic dilator”. The dilating force of the internal nasal dilator is a support force or a resilient force of the intranasal stents, and the central portion of the internal nasal dilator does not provide a resilient force. The central portion of this type of internal nasal dilator can provide a fixation function, which facilitates stable wearing. In addition, the effective area of the dilating force of the internal nasal dilator is usually larger than that of the first type of dilator, thereby reducing the pressure and irritation on a nasal passage inner wall. In summary, the overall performance of the second type of internal nasal dilator is superior to that of the first type of internal nasal dilator. Intranasal stents of this type of internal nasal dilator may include an annular stent, U-shaped stent, and an H-shaped stent.

The invention patent entitled Nasal dilator (Application Publication No.: U.S. Pat. No. 6,978,781B1), the invention patent entitled Nasal dilator (Application Publication No.: U.S. Pat. No. 5,895,409A), and the invention patent entitled Nasal dilator (Application Publication No.: US20060266367A1) have an annular intranasal stent. An outer wall of the intranasal stent conforms to and supports the nasal passage inner wall, a central axis of an annular structure is parallel to the nasal passages, and respiratory airflow passes through a vertically penetrating inner cavity of the annular structure. After the annular stent is inserted into the nasal passage to a certain depth, the annular stent provides a 360-degree support force and dilating force to a nasal passage inner wall, and the annular stent has a large contact area with the nasal cavity and is therefore comfortable to wear. When made of pliable materials, the intranasal stent in the dilator can effectively adapt to nasal passages of circular shapes, elliptical shapes, elongated shapes, or irregular shapes, and provide a uniform and gentle dilating force to the nasal passages. In addition, large contact surface areas between the intranasal stent and the nasal passage inner wall can enhance the frictional force between the two, thereby improving the fixation of the internal nasal dilator. Moreover, the annular structure is also conducive to enhancing the radial supporting and dilating effects of the intranasal stent. Finally, the annular stent does not span the nasal passages, and has the lowest respiratory resistance among all intranasal stents. A disadvantage of the annular stent is a fixed perimeter of the annular stent, making the size nonadjustable, and a number of size models need to be arranged to adapt to nasal passages of different sizes. As a single-use product, the annular stent has high trial costs and occupies more shelf space, and as a result has reduced versatility and practicability. In addition, two annular stents with identical perimeters are only applicable to bilaterally symmetrical nasal passages, and are not applicable to users with asymmetrical nasal passages on two sides such as deviation of the nasal septum.

The invention patent entitled MULTI-LAYER INTERNAL NASAL DILATOR WITH TUBULAR EXPANDERS AND COMPOUND DELIVERY PROTRUSIONS (Application Publication No.: U.S. Pat. No. 8,491,622B2) and the invention patent entitled nasal cavity dilator (Application Publication No.: CN108348350A) have a U-shaped stent. A U-shaped stent is formed by removing a portion of the sidewall of an annular stent. An opening in the sidewall of the U-shaped stent makes the perimeter of the U-shaped stent adjustable, thereby enhancing the adaptability to nasal passages of different sizes, and achieving applicability to users with asymmetrical nasal passages on two sides. The disadvantages of the U-shaped stent are as follows. First, the opening structure makes the dilating force of the sidewall of the U-shaped stent smaller than that of the annular stent. Second, due to the opening structure, the effective area of the dilating force of the U-shaped stent is smaller than that of the annular stent, and an edge region at the opening position may irritate a nasal passage inner wall. Third, the U-shaped stent has a limited size adjustment range, and a number of size models still need to be arranged to adapt to nasal passages of different sizes, and the U-shaped stent is also not applicable to users with severely asymmetrical nasal passages on two sides. In summary, the U-shaped stent modestly improves versatility compared with the annular stent but compromises some dilating force and comfort.

The invention patent entitled Internal nasal dilator and delivery mechanism (Application Publication No.: U.S. Pat. No. 7,055,523B1) and the invention patent entitled Device for improving air flow through a nasal cavity during physical activity such as sporting pursuits (Application Publication No.: US20150196420A1) have an H-shaped intranasal stent. The H-shaped intranasal stent is formed by two substantially parallel plate-shaped structures and a support arm connecting the two. As seen from the top, the three generally have an “H” shape. During wearing, one plate-shaped structure conforms to the nasal septum, the other plate-shaped structure conforms to and supports the nasal ala inner wall, and the two are connected by one or more support arms that span nasal passages. The advantage of the H-shaped stent is a strong localized support force for the nasal alae. The disadvantages of the H-shaped stent are as follows: First, the H-shaped stent has small contact areas with a nasal passage inner wall, tends to irritate the nasal passages, and provides less comfort than the annular and U-shaped stents. Second, the core support arm of the H-shaped stent spans the central portions of the nasal passages, leading to a larger resistance to the respiratory airflow than the annular and U-shaped stents. Third, limited by the support arm, the H-shaped stent has poor versatility, and a number of size models need to be arranged to adapt to nasal passages of different sizes.

In addition, according to the structure, the internal nasal dilator may include an integral structure and a separable structure. Existing internal nasal dilators usually use an integral structure, that is, are formed by injection molding or compression molding in one step in a mold using a single material. The reason for this lies in that as the internal nasal dilator has a small size, a separable structure struggles to ensure the reliability of connection, which may cause inhalation and suffocation risks upon disassembly, and an integral structure has a relatively low risk of disassembly. However, the integral structure has the following disadvantages: First, the integral structure can only use a single material, and cannot achieve optimal performance of the internal nasal dilator. For example, when a soft material is used, the central portion of the internal nasal dilator struggles to clamp the nasal septum, resulting in a significant reduction in the fixation function of the internal nasal dilator. When a rigid material is used, the intranasal stent of the internal nasal dilator tends to irritate the nasal passage, and has significantly reduced conformability to nasal passages of different shapes. Second, the integral structure also renders the intranasal stent undetachable and unreplaceable. Third, the manufacturing difficulty is high, high costs are required, and it is not easy to add more functional structures. Compared with the deficiencies of the integral structure, once the connection reliability of the separable structure can be ensured, numerous limitations of the integral structure can be overcome, thereby significantly enhancing the performance of the internal nasal dilator.

Moreover, some internal nasal dilators, for example, the foregoing internal nasal dilators in U.S. Pat. No. 6,863,066B2 and US20150196420A1, have a size adjustment function. However, both have particularly limited performance. The internal nasal dilator in U.S. Pat. No. 6,863,066B2 is an external nasal elastic dilator, which has inherent disadvantages of this type of dilator such as poor wearing stability, low comfort, and respiratory obstruction. In addition, the internal nasal dilator adjusts the length of an annular metal wire in a manner of inserting the annular metal wire into a tubular structure, and a size adjustment range is small, and size locking is also not reliable. The internal nasal dilator in US20150196420A1 has an H-shaped stent. The intranasal stent includes two plate-shaped structures that conform to the nasal septum and the nasal ala inner wall respectively and one arc-shaped support rib. As an innovation, the intranasal stent of the internal nasal dilator further includes one annular belt used for adjusting the size and providing an auxiliary support force. In view of the fact that the internal nasal dilator has adhered to the conventional design ideas of an H-shaped stent and an integral structure, the internal nasal dilator has the following limitations: First, a size adjustment range is small, and a number of size models still need to be arranged. On one hand, the support rib of the intranasal stent has a limited elastic expansion range, which limits the adjustment range of the annular belt. On the other hand, in an adjustment process, the annular belt pulls the support rib to twist toward one side, and the torque of the support rib further limits the adjustment range of the annular belt. Second, the internal nasal dilator has “an integral structure formed in one step in a mold”, and therefore has inherent disadvantages of the integral structure described above. Third, as an auxiliary structure, the annular belt of the intranasal stent does not have the advanced performance of an independent annular stent. Because the dilating force of the intranasal stent of the internal nasal dilator mainly comes from the support rib, the dilating force is mainly applied to the nasal ala position. Therefore, the annular belt of the intranasal stent can neither autonomously provide a 360-degree dilating force to a nasal passage inner wall like an annular stent nor conform to the nasal passage inner wall, and therefore cannot obtain optimal matching and fixation with the nasal passages.

In summary, existing technologies still fail to achieve core performance of an internal nasal dilator such as a dilation function, wearing stability, versatility, and comfort.

### SUMMARY OF THE INVENTION

The present invention is the foregoing “internal nasal elastic dilator”, uses a separable plug-in structure, and includes an annular intranasal stent with an adjustable size, so that one size model can match nasal passages of all sizes. An objective of the present invention is to comprehensively improve the dilation function, wearing stability, versatility, and comfort of an internal nasal dilator.

To achieve the foregoing objective, the present invention provides an internal nasal dilator, where the internal nasal dilator includes one nose clip and two intranasal stents;
the nose clip includes one substantially U-shaped or C-shaped nose clip body and two fixing clips that are symmetrically arranged and are integrally connected to distal ends of two branches of the nose clip body; and
each of the intranasal stents self-expands into a flat ribbon-shaped structure, is detachably connected and fixed to each of the fixing clips of the nose clip, and coils to form an annular structure with an adjustable perimeter.

As an improvement to the foregoing internal nasal dilator, an axis of the annular structure is substantially parallel to an axis of a nasal passage in which the annular structure is located.

As an improvement to the foregoing internal nasal dilator, the fixing clip of the nose clip includes an annular clip frame, a vertically penetrating fixing hole is provided in the clip frame, and a clip opening is provided on an outer side of the fixing hole;
the intranasal stent includes a stent body and a stent tail;
the stent tail is integrally connected to an end of the stent body, the stent tail includes an annular frame, the frame is provided with an insertion hole penetrating the frame, and the frame of the stent tail surrounds and is fixed to the clip frame; and
a portion of the stent body is fixed in the fixing hole and cooperates with the fixing clip to adjust and fix the perimeter of the annular structure.

As an improvement to the foregoing internal nasal dilator, the fixing clip of the nose clip includes an annular clip frame, a vertically penetrating fixing hole is provided in the clip frame, the fixing hole includes an inner hole and an outer hole, and a partition is provided between the inner hole and the outer hole;
the intranasal stent includes a stent body and a stent tail;
the stent tail is integrally connected to an end of the stent body, a tail end is provided at an end of the stent tail that is away from the stent body, a lateral perimeter of the tail end is greater than a lateral perimeter of the stent tail, a protruding tail tooth is provided at an end of the stent tail that is close to the stent body, the stent tail is inserted into the outer hole, and the tail end and the tail tooth are exposed from two ends of the outer hole to fix the intranasal stent and the nose clip to each other; and
a portion of the stent body is fixed in the inner hole and cooperates with the fixing clip to adjust and fix the perimeter of the annular structure.

As an improvement to the foregoing internal nasal dilator, a plurality of clip teeth arranged at fixed intervals are further provided at the stent body of the intranasal stent and cooperate with the fixing clip to adjust and fix the perimeter of the annular structure.

As an improvement to the foregoing internal nasal dilator, a serrated or undulating structure is arranged on an upper side and/or a lower side of the stent body of the intranasal stent and cooperates with the fixing clip to adjust and fix the perimeter of the annular structure.

As an improvement to the foregoing internal nasal dilator, the fixing clip of the nose clip includes an annular clip frame, and a vertically penetrating fixing hole is provided in the clip frame;
the intranasal stent includes a stent body; and
self-fixing devices with an adjustable fixing position extend outward from two ends of the stent body and form the annular structure after coiled overlapping fixation.

As an improvement to the foregoing internal nasal dilator, the nose clip and the intranasal stent are made of different materials, the nose clip is made of a rigid elastic material, and the intranasal stent is made of a soft elastic material.

As an improvement to the foregoing internal nasal dilator, at least one ribbon-shaped support spoke with two ends integrally connected to an inner wall of an intranasal stent is arranged in the intranasal stent.

As an improvement to the foregoing internal nasal dilator, a structure used for accommodating a compound is arranged on an inner side of the annular structure of each intranasal stent, and is capable of releasing the compound into the nasal passage or absorbing and filtering specific substances from airflow in the nasal passage.

The internal nasal dilator of the present invention compactly and reliably combines the three advantageous technologies of separable structures, annular intranasal stents, and the size adjustment function, and comprehensively improves the performance of internal nasal dilators. Details are as follows.
1. A separable plug-in structure is used. The intranasal stent uses a soft elastic material, which softly conforms to a nasal passage inner wall, thereby ensuring comfort. The nose clip uses an elastic material more rigid than that of the intranasal stent, so that the nose clip stably clamps and is secured at the lower portion of the nasal septum, thereby ensuring wearing stability. In addition, the nose clip made of a rigid material is conducive to fixing the intranasal stent, thereby keeping the intranasal stent from loosening and torsional deformation, and ensuring reliable locking of the clip teeth. In addition, the intranasal stent can be conveniently detached, making it convenient for a user to replace and use intranasal stents of different functions or filling or replacing various compounds on the intranasal stent. Moreover, the separable plug-in design can disassemble a complex overall structure into a number of simple components for manufacturing, thereby significantly reducing manufacturing costs, and manufacturing an internal nasal dilator with a more complex structure.
2. The intranasal stent uses a soft and elastic annular structure to achieve maximal comfort. First, the annular stent provides 360-degree support to the nasal passage inner wall to obtain a maximum force-bearing area, thereby minimizing the pressure and irritation on the nasal passage inner wall by the intranasal stent. Second, the soft annular structure can optimally adapt to nasal passages of different shapes such as circular shapes, elliptical shapes, elongated shapes, and irregular shapes, and uniformly apply a dilating force to the nasal passages to obtain excellent comfort. Third, good conformity enhances the frictional force between the intranasal stent and the nasal passage inner wall, thereby improving wearing stability. Fourth, the annular structure can provide an excellent radial support force, so that the internal nasal dilator has excellent dilation performance. Fifth, the annular stent is located at a peripheral position of the nasal passages, and does not block intranasal airflow, so that the ventilation performance is excellent.
3. The plug-in position and the size adjustment mechanism are combined in a simple and reliable manner, and the adjustment mechanism can adjust the size of the intranasal stent within a large range, so that high versatility of one size model matching all nasal passages is achieved, and secure locking can be achieved at various size adjustment positions. In addition, the internal nasal dilator of the present invention is also applicable to users with different degrees of asymmetrical nasal passages on two sides.
4. The performance of the internal nasal dilator can be further adjusted and improved by adjusting a support angle and adding a support spoke. In addition, a new functional structure is added to the intranasal stent, various functions such as releasing a compound and absorbing and filtering specific substances from respiratory airflow can be conveniently added.

### BRIEF DESCRIPTION OF THE DRAWINGS

FIG. 1A is a side view of a nasal region of a human;

FIG. 1B is a cross-sectional side view of a nasal region of a human;

FIG. 1C is a bottom view of a nasal region of a human;

FIG. 2 is a schematic structural diagram of a first preferred embodiment according to the present invention;

FIG. 3 is a schematic structural diagram of the first preferred embodiment from another perspective according to the present invention;

FIG. 4 is a front view of the first preferred embodiment according to the present invention;

FIG. 5 is a top view of the first preferred embodiment according to the present invention;

FIG. 6 is a side view of the first preferred embodiment according to the present invention;

FIG. 7 is a bottom view of the first preferred embodiment after wearing according to the present invention;

FIG. 8 is a front view of a nose clip of the first preferred embodiment according to the present invention;

FIG. 9 is a top view of an intranasal stent of the first preferred embodiment coiled to be annular according to the present invention;

FIG. 10 is a front view of an expanded intranasal stent of the first preferred embodiment according to the present invention;

FIG. 11 is a front view of two intranasal stents of the first preferred embodiment with an included angle optimized according to the present invention;

FIG. 12 is a cross-sectional view at a position i-i′ in FIG. 4 of the first preferred embodiment after a fixing clip is optimized according to the present invention;

FIG. 13 is a schematic structural diagram of an expanded intranasal stent of the first preferred embodiment with a drug groove added according to the present invention;

FIG. 14A is a schematic structural diagram of an expanded intranasal stent of the first preferred embodiment with a release element added according to the present invention;

FIG. 14B is a schematic diagram of disassembly of a release element of an expanded intranasal stent of the first preferred embodiment with the release element added according to the present invention;

FIG. 15A is a schematic structural diagram of an expanded intranasal stent of the first preferred embodiment with a drug cartridge added according to the present invention;

FIG. 15B is a schematic structural diagram of an expanded intranasal stent of the first preferred embodiment with a drug cartridge added from another perspective according to the present invention;

FIG. 16 is a top view of the first preferred embodiment with a support spoke added according to the present invention;

FIG. 17 is a schematic structural diagram of a second preferred embodiment according to the present invention;

FIG. 18 is a schematic structural diagram of the second preferred embodiment from another perspective according to the present invention;

FIG. 19 is a front view of the second preferred embodiment according to the present invention;

FIG. 20 is a top view of the second preferred embodiment according to the present invention;

FIG. 21 is a side view of the second preferred embodiment according to the present invention;

FIG. 22 is a front view of a nose clip of the second preferred embodiment according to the present invention;

FIG. 23 is a top view of an intranasal stent of the second preferred embodiment coiled to be annular according to the present invention;

FIG. 24 is a front view of an expanded intranasal stent of the second preferred embodiment according to the present invention;

FIG. 25 is a schematic structural diagram of a third preferred embodiment according to the present invention;

FIG. 26 is a top view of the third preferred embodiment according to the present invention;

FIG. 27 is a schematic structural diagram of a nose clip of the third preferred embodiment according to the present invention;

FIG. 28 is a schematic structural diagram of an intranasal stent of the third preferred embodiment coiled to be annular according to the present invention;

FIG. 29 is a schematic structural diagram of an expanded intranasal stent of the third preferred embodiment according to the present invention; and

FIG. 30 is a side view of an expanded intranasal stent of the third preferred embodiment according to the present invention;

### REFERENCE NUMERALS

| | |
|---|---|
| 1. internal nasal dilator | 2. nose clip |
| 3. intranasal stent | 9. nasal region |
| 11. anterior direction | 12. posterior direction |
| of the nasal region | of the nasal region |
| 13. superior direction | 14. inferior direction |
| of the nasal region | of the nasal region |
| 21. fixing clip | 22. nose clip body |
| 31. stent body | 32. stent tail |
| 34. clip teeth | 35. drug groove |
| 36. release element | 37. drug cartridge |
| 38. snap fastener | 39. support spoke |
| 91. nasal septum | 92. nasal ala |
| 93. nasal bridge | 94. nostril |
| 95. nasal passage | 96. nasal passage inner wall |
| 97. nasal passage axis | 98. nasal cavity |
| 99. nasal base | |
| 211. clip frame | 212. fixing hole |
| 213. clip opening | 214. inner hole |
| 215. outer hole | 221. connecting beam |
| 222. branch | |
| 321. frame | 322. insertion hole |
| 323. tail end | 324. tail tooth |
| 325. central portion | 361. fixing tube |
| 362. drug-loading insert | 381. snap fastener plug |
| 382. snap fastener hole | |
| 2111. upper wall | 2112. lower wall |
| 2113. inner wall | 2114. outer wall |
| 2115. partition | 2116. fixing hook |

### DETAILED DESCRIPTION

The present invention is further described with reference to the accompanying drawings and embodiments.

The term “connected” as used in the present invention refers to that two tangible objects are integrally or detachably connected together. A connection manner includes, but is not limited to: bonding, welding, insertion, a threaded connection, and a buckle connection.

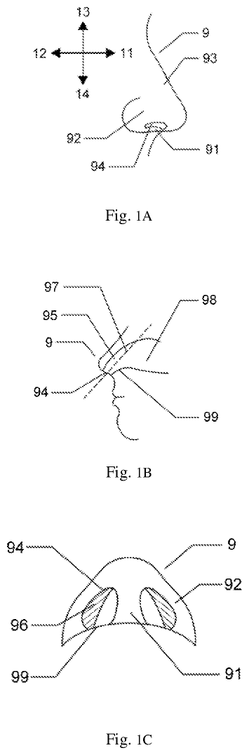

The term “axis” as used in the present invention refers to the central symmetric axis of a tubular object or columnar object. A non-standard tubular object or columnar object may be approximated as a standard tubular object or columnar object, and the axis of the standard tubular object or columnar object is used as an approximate axis of the non-standard tubular object or columnar object. For example, FIG. 1B describes a nasal passage axis 97.

The term “compound” as used in the present invention is not limited to a strict chemical definition, but shall include solid or liquid pure substances, chemical compounds, and mixtures, as well as other material forms or combinations applicable to the present invention. The compound may be pharmaceutical or non-pharmaceutical, including, but not limited to, medicinal agents and aromatic substances.

As shown in FIG. 1A, the direction orientations of the nasal region of the present invention are an anterior direction 11 of the nasal region, a posterior direction 12 of the nasal region, a superior direction 13 of the nasal region, and an inferior direction 14 of the nasal region.

As shown in FIG. 1B, for an object in a nasal passage 95, a side of the object close to a nasal passage axis 97 is an “inner side”, and a side of the object away from the nasal passage axis 97 is an “outer side”.

To resolve the deficiencies of an existing internal nasal dilator, this application makes improvements in the following aspects: First, a separable structure is used while the connection reliability is ensured. Second, an annular intranasal stent is used, and a soft elastic material is used. Third, an adjustable range of a size adjustment mechanism is expanded, thereby achieving higher versatility.

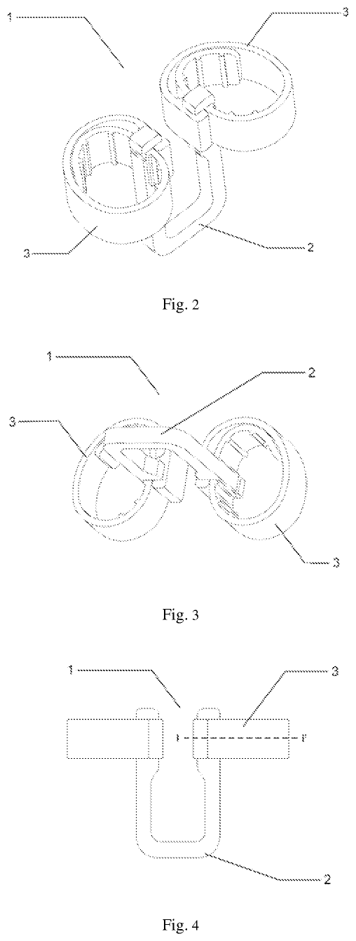

As shown in FIG. 2, in an internal nasal dilator 1 of the present invention, in addition to a nose clip 2, a nose clip body 22, and a connecting beam 221, all the remaining components are two or two groups of same components that are symmetrically arranged for two nasal passages. Therefore, for the description of the internal nasal dilator 1 of the present invention, apart from the nose clip 2, the nose clip body 22, and the connecting beam 221, each of the remaining components is one or one group in the two or two groups of same components that are symmetrically arranged.

As shown in FIG. 1A, FIG. 1B, and FIG. 1C, the internal nasal dilator 1 of the present invention is applied to a nasal region 9 of a human. The nasal region 9 is a visually prominent part of the facial protrusion, including a nasal septum 91, a nasal ala 92, a nasal bridge 93, and a nasal base 99, as well as two nasal passages 95 defined thereby. Exits of the two nasal passages 95 are two nostrils 94. A nasal passage inner wall 96 is formed by inner side surfaces of the sites surrounding the nasal passage 95. In the present invention, the nasal passage 95 is only limited to the nasal vestibule included in the nasal region 9, and does not include a nasal cavity 98.

### Embodiment 1

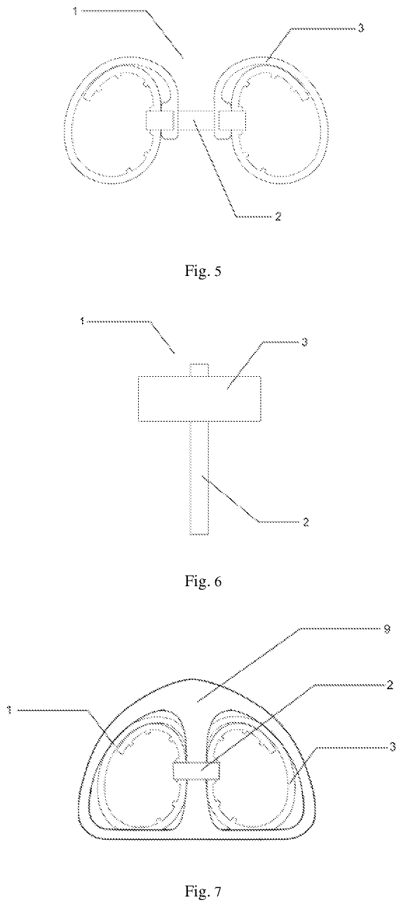

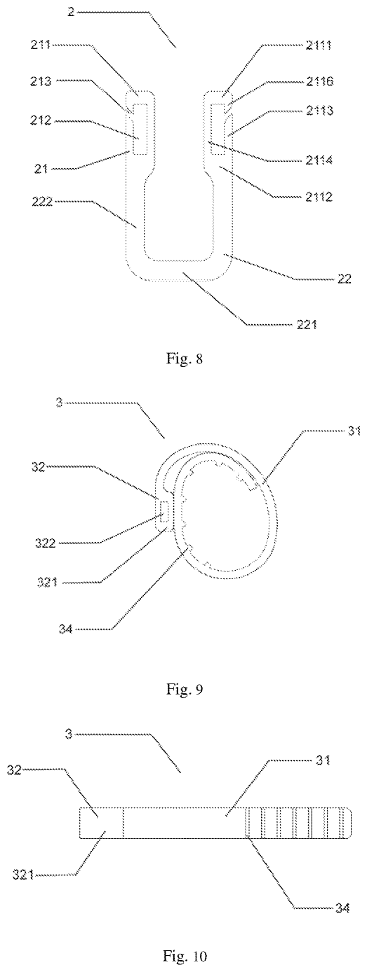

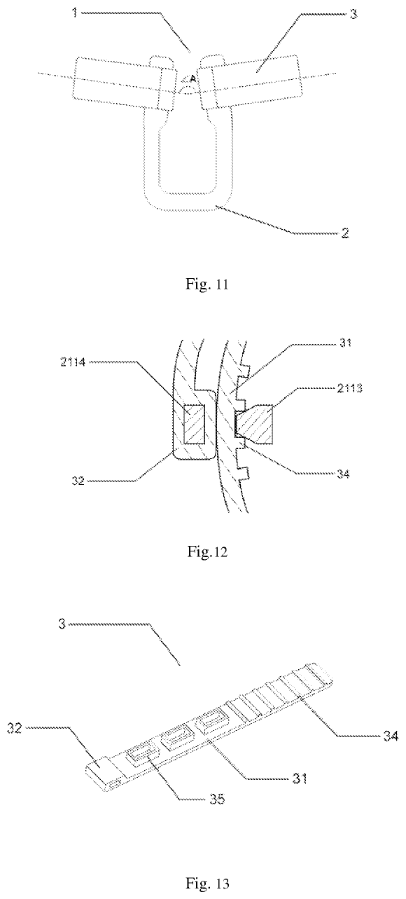

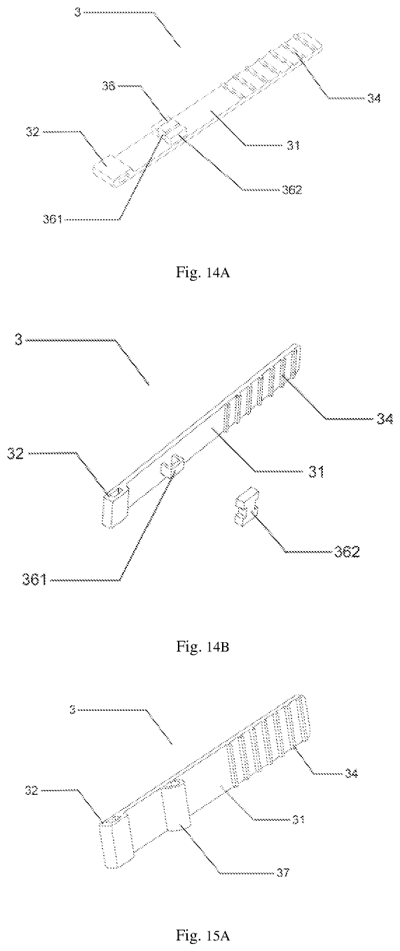

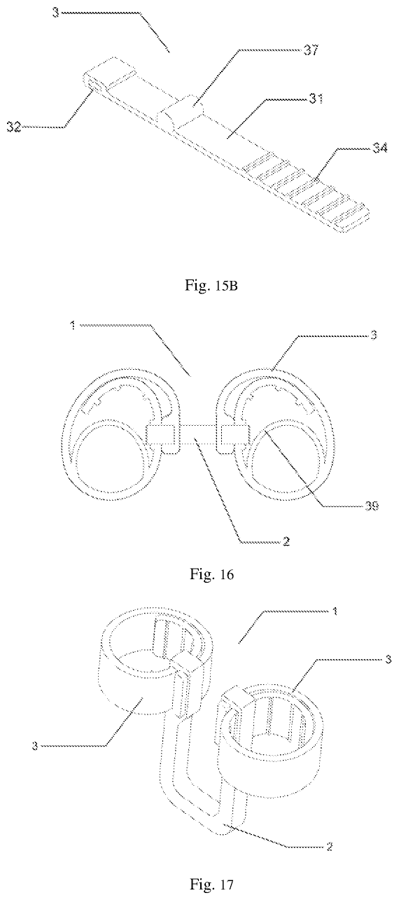

FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14A, FIG. 14B, FIG. 15A, FIG. 15B, and FIG. 16 show a first embodiment of the present invention. As shown in FIG. 2, FIG. 3, FIG. 4, FIG. 5, FIG. 6, and FIG. 7, the present invention provides an internal nasal dilator 1 used for dilating the nasal passage 95 in the nasal passage 95. The internal nasal dilator 1 includes one nose clip 2 and two intranasal stents 3 that are symmetrically arranged. The internal nasal dilator 1 is a separable plug-in structure. The nose clip 2 and the two intranasal stents 3 are components that are independent of each other. Two ends of the nose clip 2 are detachably connected to the two intranasal stents 3 in a plug-in manner. As an optimization, the nose clip 2 and the two intranasal stents 3 may be made of different materials, thereby achieving optimal performance of the internal nasal dilator 1.

As shown in FIG. 8, the nose clip 2 is a U-shaped or C-shaped integral structure, including: two symmetrically arranged fixing clips 21 and one nose clip body 22. Each fixing clip 21 includes a clip frame 211, a fixing hole 212, and a clip opening 213. The fixing clip 21 is used for connecting and fixing the intranasal stent 3, and cooperating with the intranasal stent 3 to achieve a size adjustment function. Specific manners of assembly and size adjustment are described below in detail. The nose clip body 22 is a U-shaped or C-shaped structure, and includes one connecting beam 221 and two symmetrically arranged branches 222. The two fixing clips 21 are respectively integrally connected to upper portions of the two branches 222.

As shown in FIG. 7, during wearing, the nose clip 2 half surrounds a lower portion of the nasal septum 91. In this case, the connecting beam 221 is located below the nasal septum 91, and the two branches 222 are fully or partially inserted into two nostrils 94 respectively, so that the two fixing clips 21 are respectively located in two nasal passages 95, and the two fixing clips 21 are connected in a plug-in manner to the two intranasal stents 3 that are symmetrically arranged in the two nasal passages 95. In this case, inner sides of the two branches 222 and/or the two fixing clips 21 conform to the nasal septum 91, and the nose clip 2 half surrounds a wide lower end of the nasal septum 91, making it difficult for the nose clip 2 to detach from the nasal septum 91. As an optimization, the nose clip 2 applies a clamping force to the nasal septum 91. In this case, the two fixing clips 21 and/or the two branches 222 clamp the nasal septum 91, to fix the nose clip 2 on the nasal septum 91 more securely. As a further optimization, for two insertion pins of the nose clip 2 formed by the two fixing clips 21 and the two branches 222, one boss facing the nasal septum is symmetrically arranged on a side of each of the insertion pins that faces the nasal septum. A spacing between the two bosses is less than a width of the lower end of the nasal septum 91. During wearing, the two bosses are located above a wide position of the nasal septum 91 to keep the nose clip 2 from falling off the nasal septum. As a further optimization, surfaces of the fixing clips 21 and/or the branches 222 that conform to the nasal septum 91 are appropriately enlarged to reduce the pressure of the nose clip 2 on the nasal septum 91.

In summary, the nose clip 2 can achieve the following functions: connecting and fixing the intranasal stent 3; fixing the internal nasal dilator 1 on the nasal septum 91; and cooperating with the intranasal stent 3 to achieve the size adjustment function.

To ensure that the functions of the nose clip 2 are achieved, the nose clip 2 is usually made of a rigid elastic material to obtain the following benefits: first, the nose clip 2 is fixed on the nasal septum 91 more reliably; second, the intranasal stent 3 is connected and fixed more securely, so that the intranasal stent 3 remains in a suitable posture and support angle during wearing; and third, the following clip teeth 34 are fixed more reliably to keep the clip teeth 34 from being disengaged.

As shown in FIG. 10, before assembly, the intranasal stent 3 self-expands into a flat ribbon-shaped structure, and has a large width ranging from 3 mm to 15 mm to increase a contact area between the intranasal stent 3 and the nasal passage inner wall 96, thereby reducing the pressure caused by a dilating force. In addition, the large width is also conducive to fixing the intranasal stent 3 on the nose clip 2 more stably, and remaining in a suitable posture and support angle during wearing. In addition, the width of the expanded intranasal stent 3 is the same as the height of the fixing hole 212, so that after being mounted, the intranasal stent 3 is stably fixed in the fixing hole 212. As an optimization, the intranasal stent 3 may have different widths at different positions to achieve a specific function. For example, the width of the intranasal stent 3 is increased at the nasal ala to enlarge a contact area between the intranasal stent 3 and the nasal ala, thereby improving comfort. For another example, in other embodiments, a serrated or undulating structure is arranged on the upper side and/or a lower side of the annular intranasal stent 3, and the intranasal stent 3 cooperates with an upper wall 2111 or a lower wall 2112, to enable the upper wall 2111 or the lower wall 2112 to be secured at the trough of the serrated or undulating structure, thereby adjusting and fixing the perimeter of the annular structure of the intranasal stent 3. The intranasal stent 3 has a small thickness ranging from 0.5 mm to 3 mm to minimize the resistance of the intranasal stent 3 to respiratory airflow and improve the matching capability of the intranasal stent 3 to nasal passages of different shapes. As an optimization, the intranasal stent 3 may have different thicknesses at different positions to achieve a specific function.

As shown in FIG. 9, after assembly, the ribbon-shaped structure of the intranasal stent 3 is coiled to form an annular structure, so that the intranasal stent 3 has the following benefits of an annular stent: first, the intranasal stent 3 applies a 360-degree dilating force to the nasal passage inner wall to reduce the pressure of the dilating force to the minimum to improve comfort; second, the annular structure provides an excellent radial support force, so that the dilating performance is excellent; third, the intranasal stent 3 can deform to automatically match nasal passages with cross-sections of circular shapes, elliptical shapes, elongated shapes, or irregular shapes, to obtain optimal conformity and comfort; fourth, the annular stent conforms to the nasal passage inner wall and does not have a structure or component located at the central portion of the nasal passage, and therefore the intranasal stent 3 has an extremely small resistance to respiratory airflow; and fifth, the contact area between the intranasal stent 3 and the nasal passage inner wall is large, so that the frictional force between the intranasal stent 3 and the nasal passage inner wall can be increased, thereby fixing the intranasal stent 3 in the nasal passage 95. In this case, the internal nasal dilator 1 has the nasal septum 91 and the nasal passage 95 as two fixing regions, thereby achieving excellent fixation.

As a further optimization, the intranasal stent 3 is usually made of an elastic material softer than that of the nose clip 2 to increase the matching degree between the intranasal stent 3 and nasal passages 95 of different shapes, thereby reaching the maximum contact surface area, to obtain optimal comfort and fixation.

As shown in FIG. 9 and FIG. 10, each intranasal stent 3 is an integral structure, including one stent body 31, one stent tail 32, and a plurality of clip teeth 34. The stent body 31 is a ribbon-shaped structure, an end of which is integrally connected to the stent tail 32. The clip teeth 34 are arranged on an inner surface of the stent body 31, and the number of the clip teeth 34 may be increased or reduced according to an actual requirement, and is preferably 7. The stent tail 32 includes a frame 321 and an insertion hole 322, and is connected to the fixing clip 21 in a plug-in manner. The stent tail 32 is fixed on the nose clip 2 and becomes the first fixing position of the intranasal stent 3 on the fixing clip 21. The stent body 31 is coiled to form an annular structure. A portion of the stent body 31 is detachably fixed in the fixing hole 212 and becomes the second fixing position of the intranasal stent 3 on the fixing clip 21, to fix the intranasal stent 3 as an annular structure together with the stent tail 32. To adjust the size of the intranasal stent 3, a user may select different clip teeth 34 to be secured on the clip frame 211 as the second fixing position, to enable the intranasal stent 3 to match nasal passages 95 of different sizes.

As shown in FIG. 2, FIG. 3, FIG. 4, FIG. 5, FIG. 6, FIG. 8, FIG. 9, and FIG. 10, an assembly method of the internal nasal dilator 1 is as follows: An upper portion of the fixing clip 21 is inserted into the insertion hole 322, and an inner side of the frame 321 is pressed to elastically dilate outward to be sleeved over the upper portion of the fixing clip 21. Next, a lower edge of the frame 321 is inserted into the fixing hole 212 through the clip opening 213, to enable the stent tail 32 to surround and to be fixed on an outer wall 2114. Subsequently, the ribbon-shaped intranasal stent 3 is coiled to form an annular structure. A suitable position on the stent body 31 is selected based on the size of the nasal passage of the user. The lower edge of the stent body 31 at this position is inserted through the clip opening 213 and is fixed in the fixing hole 212. In this case, a total thickness of the frame 321 and the stent body 31 stacked in the fixing hole 212 is equal to or slightly greater than the width of the fixing hole 212, to enable the fixing hole 212 to tightly surround or clamp the two, and clip teeth 34 (the fourth and fifth clip teeth counting from an end of the stent body 31 that is away from the stent tail 32 in FIG. 5) at this position are firmly secured on two sides of an inner wall 2113. The position of the stent body 31 that is fixed by the fixing hole 212 cooperates with the stent tail 32, to enable the intranasal stent 3 to remain an annular structure. In this case, the perimeter of the annular structure is the length of the intranasal stent 3 between the two fixing positions. In addition, a section from the end of the stent body 31 that is away from the stent tail 32 to the fixing hole 212 is located on the inner side of the annular structure, and therefore does not irritate the nasal passage inner wall 96, and can support the intranasal stent 3 from the inner side of the annular structure, thereby enhancing the dilating force of the intranasal stent 3. Moreover, after the frame 321 and the stent body 31 are inserted into the fixing hole 212, a wedge-shaped structure of a fixing hook 2116 safely locks the two in the fixing hole 212, to keep the two from being disengaged from the fixing hole 212. Finally, the user selects a different position of the stent body 31 to be fixed in the fixing hole 212, and clip teeth 34 corresponding to the position are secured on two sides of the inner wall 2113, so that the perimeter of the annular intranasal stent 3 can be adjusted and locked, thereby achieving the size adjustment function. If the nasal passages of a user on two sides have different sizes, the two symmetrically arranged intranasal stents 3 may be adjusted to different sizes to match the nasal passages.

As a further optimization, as shown in FIG. 12, the shape of the cross-section of the inner wall 2113 is adjusted to reduce the width of a side of the inner wall 2113 that is close to the fixing hole 212, so that more clip teeth 34 with smaller spacings can be arranged on the stent body 31, thereby achieving more accurate adjustment.

As a further optimization, the clip teeth 34 are not arranged on the intranasal stent 3, and the total thickness of the frame 321 and the stent body 31 stacked in the fixing hole 212 is greater than the width of the fixing hole 212. In this case, the frame of the fixing hole 212 clamps and presses the two, thereby fixing the annular structure of the intranasal stent 3.

As a further optimization, the clip teeth 34 are not arranged in the annular intranasal stent 3, and the serrated or undulating structure is arranged on an upper side and/or a lower side of the stent body 31, and cooperates with the upper wall 2111 or the lower wall 2112, so that the upper wall 2111 or the lower wall 2112 is secured at the trough of the serrated or undulating structure, thereby adjusting and fixing the perimeter of the annular structure of the intranasal stent 3. As a further optimization, the shape of the cross-section of the upper wall 2111 or the lower wall 2112 is adjusted to reduce the width of a side of the upper wall 2111 or the lower wall 2112 that is close to the fixing hole 212, so that more serrated or undulating structures with smaller spacings can be arranged on the stent body 31, thereby achieving more accurate adjustment.

The size adjustment function in this embodiment has the following characteristics and benefits: First, the versatility is high. The intranasal stent 3 is an independent structure, and any position on the stent body 31 may be selected as the second fixing position other than the stent tail 32, so that the adjustment range can be large, thereby achieving that one size model matches nasal passages of all sizes. Second, the locking is reliable. In an assembly process in this embodiment, the clip teeth 34 do not need to pass through the fixing hole 212 as in Embodiment 2. Therefore, clip teeth 34 with a large height can be used, thereby improving the reliability of locking. Third, the structure is simple and integrated. The connection position between the nose clip 2 and the intranasal stent 3 has a simple structure, is safe and reliable, has a small resistance to respiratory airflow, and combines a plug-in fixing function and a size adjustment function, so that this embodiment has advantages of a reliable separable structure and size adjustment within a large range.

After assembly, as shown in FIG. 4, the annular structures of the two intranasal stents 3 are substantially located in a same plane, and the plane is substantially perpendicular to a nasal passage axis 97. As an optimization, postures of the two intranasal stents 3 in the nasal passages 95 can be adjusted by changing the shape of the nose clip 2 or the angle of the fixing hole 212, thereby obtaining optimal dilation. For example, FIG. 11 shows an optimized structure, in which the nose clip 2 is C-shaped, and the two fixing clips 21 form a certain included angle, so that an included angle ∠A between planes in which the annular structures of the two intranasal stents 3 are located ranges from 90° to 180°.

During wearing, as shown in FIG. 7, the user inserts two annular intranasal stents 3 into the two nasal passages 95 respectively. In this case, the axis of the annular intranasal stent 3 is parallel to the nasal passage axis 97, the outer wall of the intranasal stent 3 gradually conforms to and supports the nasal passage inner wall 96, and respiratory airflow passes through the vertically penetrating cavity of the intranasal stent 3. The intranasal stent 3 continues to be pushed deep into the nasal passage until a satisfactory dilating position is reached. The nose clip 2 is pushed upward synchronously to enable the nose clip 2 to half surround and be fixed at the lower portion of the nasal septum 91. In this case, the outer wall of the intranasal stent 3 fully conforms to and supports the nasal passage inner wall, and the frictional force between the two enables the intranasal stent 3 to be fixed in the nasal passage.

As a further optimization, through the separable plug-in structure in this embodiment, at least one boss, recess, or an additional structure of a barrel shape, a tubular shape, a sheet shape or another shape can be conveniently arranged on the inner surface of the stent body 31 integrally or in a bonding, plug-in, or another manner, and a compound is arranged on the surface of or inside the additional structure. Through the foregoing structure, the internal nasal dilator 1 in this embodiment may have additional functions such as releasing a medicinal agent and absorbing and filtering specific substances from airflow. Three specific optimization cases are provided below.

As shown in FIG. 13, three protruding box-shaped drug grooves 35 are integrally arranged on the inner surface of the stent body 31. A jelly-like or solid compound is added into the drug grooves 35. The jelly-like or solid compound remains in the drug grooves 35. After assembly, the drug grooves 35 face the inner side of the annular structure. When respiratory airflow passes through the inner cavity of the annular structure, the drug grooves 35 can release the compound into inhalation airflow, or absorb and filter specific substances in inhalation airflow.

As shown in FIG. 14A and FIG. 14B, one release element 36 is arranged on the inner surface of the stent body 31. After assembly, the release element 36 is located on the inner side of the annular structure. The release element 36 is formed by one fixing tube 361 integrally arranged on the inner surface of the stent body 31 and one independent drug-loading insert 362. The fixing tube 361 is an elastic tubular structure, the drug-loading insert 362 is an H-shaped structure with two wide ends and a narrow central portion, and the drug-loading insert 362 may be inserted and fixed in the fixing tube 361. The drug-loading insert 362 is a porous structure, or may be a hollow structure with the outer wall provided with an opening, and can absorb or load a compound, and release the compound into the inhalation airflow.

As shown in FIG. 15A and FIG. 15B, one drug cartridge 37 is integrally arranged on the inner surface of the stent body 31. The drug cartridge 37 is a semicircular barrel structure with one end closed and the other end open. After assembly, the drug cartridge 37 is located on the inner side of the annular structure, and is filled with a compound inside, and has an opening facing the deep region of the nasal passage. When respiratory airflow passes through the inner side of the annular structure, the drug cartridge 37 may release the compound into the inhalation airflow.

As a further optimization, as shown in FIG. 16, to enhance the support force of the intranasal stent 3, at least one support spoke 39 may be arranged on the inner surface of the intranasal stent 3. The support spoke 39 is an arc-shaped ribbon-shaped structure, and has two ends integrally connected to the stent body 31. During wearing, the stent body 31 elastically deforms under the pressure of the nasal passage inner wall 96, the support spoke 39 elastically deforms synchronously, and the resilient force of the support spoke 39 enhances the dilating force of the intranasal stent 3 on the nasal passage 95.

In summary, this embodiment compactly and reliably combines the three advantageous technologies of separable structures, annular intranasal stents and size adjustment, and comprehensively improves the performance of internal nasal dilators 1.

### Embodiment 2

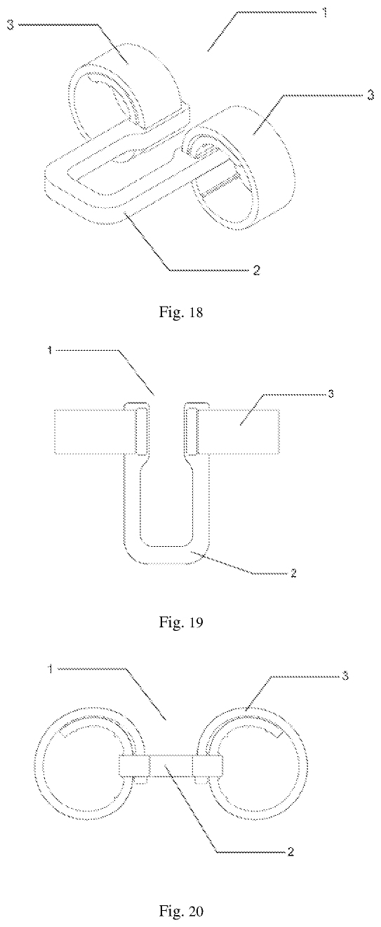

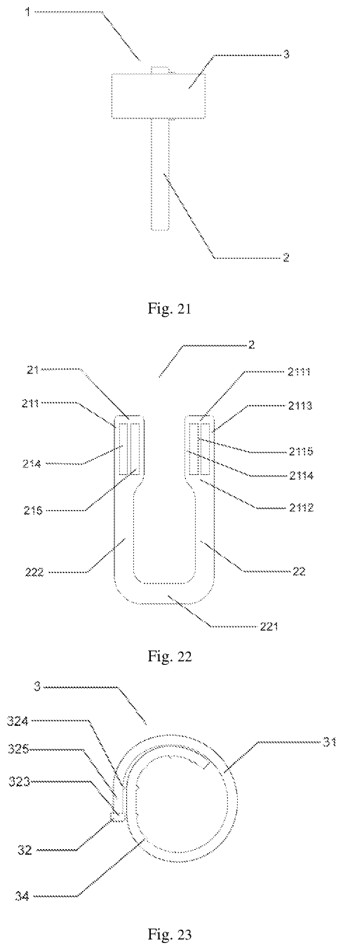

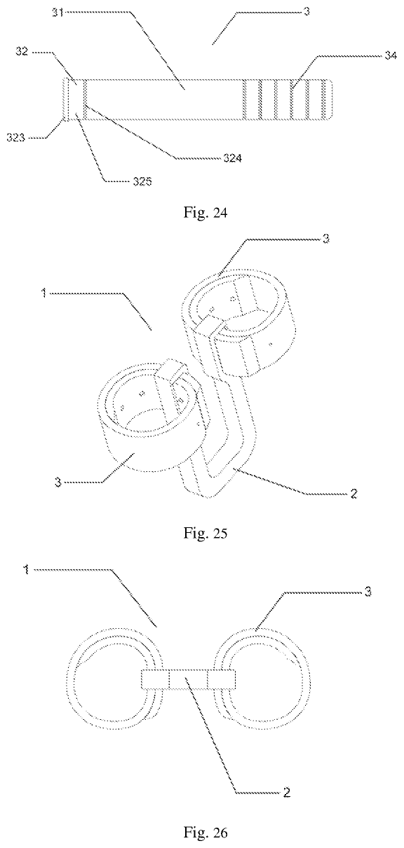

FIG. 17, FIG. 18, FIG. 19, FIG. 20, FIG. 21, FIG. 22, FIG. 23, and FIG. 24 show a second preferred embodiment of the present invention. The overall structure of the internal nasal dilator 1 in this embodiment is similar to that in the first embodiment. A special part of this embodiment lies in that the fixing clip 21 and the stent tail 32 are slightly different from the corresponding structures in the first embodiment.

As shown in FIG. 22, the fixing clip 21 includes a clip frame 211, an inner hole 214, and an outer hole 215. The inner hole 214 is used for fixing a region of the stent body 31 in which the clip teeth 34 are arranged. The outer hole 215 is used for fixing the stent tail 32. The frame 211 surrounds and wraps a part of the intranasal stent 3 that is located in the fixing clip 21. The frame 211 includes an upper wall 2111, a lower wall 2112, an inner wall 2113, an outer wall 2114, and a partition 2115.

As shown in FIG. 23 and FIG. 24, the intranasal stent 3 is an integral structure, including a stent body 31, a stent tail 32, and a plurality of clip teeth 34. The stent body 31 is a ribbon-shaped structure, an end of which is integrally connected to the stent tail 32. The clip teeth 34 are arranged on an inner surface of the stent body 31, and the number of the clip teeth 34 may be increased or reduced according to an actual requirement, and is preferably 6. An end of the stent body 31 that is away from the stent tail 32 has a gradually decreasing width, thereby facilitating the smooth insertion of the stent body 31 into a hole. The stent tail 32 connects and fixes the intranasal stent 3 on the fixing clip 21, and includes a tail end 323, a tail tooth 324, and a central portion 325 located between the two. The length of the central portion 325 is equal to the depth of the outer hole 215. The size of the tail end 323 is greater than that of the outer hole 215, thereby keeping the tail end 323 from passing through the outer hole 215. The shapes and sizes of the cross-sections of the stent body 31 and the central portion 325 are substantially the same as those of the inner hole 214 and the outer hole 215, thereby ensuring that the two positions can be passed through and stably fixed in the two holes.

As shown in FIG. 17, FIG. 18, FIG. 19, FIG. 20, FIG. 21, FIG. 22, FIG. 23, and FIG. 24, an assembly method of the internal nasal dilator 1 in this embodiment is as follows: An end of the stent body 31 that is away from the stent tail 32 is inserted into and passed through the outer hole 215, and next, the entire stent body 31 is passed through the outer hole 215, until the tail tooth 324 is also passed through the outer hole 215. In this case, the tail end 323 and the tail tooth 324 are respectively secured on two sides of the partition 2115, and the central portion 325 is located in the outer hole 215, so that the tail end 323 is fixed on the fixing clip 21 and becomes the first fixing position of the intranasal stent 3 on the fixing clip 21. Subsequently, the stent body 31 is coiled to form an annular structure, and an end of the stent body 31 that is away from the stent tail 32 is passed through the inner hole 214. Based on the size of the nasal passage of the user, an appropriate position of the stent body 31 is fixed in the inner hole 214 and becomes the second fixing position of the intranasal stent 3 on the fixing clip 21. The fixing position and the stent tail 32 make the intranasal stent 3 remain an annular structure. In this case, the clip teeth 34 (the fourth and fifth clip teeth counting from an end of the stent body 31 that is away from the stent tail 32 in FIG. 20) on two sides of the fixing position are secured on two sides of the inner wall 2113 to lock the size of the intranasal stent 3.

As a further optimization, the thickness of the end of the stent body 31 that is away from the stent tail 32 gradually decreases toward a distal end, so that the end of the stent body 31 that is away from the stent tail 32 can be inserted into the inner hole 214 and the outer hole 215 more easily, especially when the widths of the inner hole 214 and the outer hole 215 are less than the thicknesses of the stent body 31 and the central portion 325. In an assembly process, a thinner end of the stent body 31 easily guides the intranasal stent 3 to enter the two holes, to enable the two holes to clamp, press, and fix the intranasal stent 3. In this case, even if the clip teeth 34 are not arranged on the intranasal stent 3, fixation can be achieved through the foregoing clamping force.

In addition, various optimizations made in the first embodiment and the wearing method in the first embodiment are also applicable to this embodiment.

Compared with the first embodiment, the advantages of this embodiment lie in that the clip opening 213 does not need to be arranged on the nose clip 2. During use, the intranasal stent 3 does not easily fall off from the nose clip 2. The weakness of this embodiment lies in that during assembly the clip teeth 34 and tail tooth 324 need to pass through the inner hole 214 and/or the outer hole 215, and therefore the height of the clip teeth is restricted, leading to proneness to disengagement and a size locking effect inferior to that of the first embodiment.

### Embodiment 3

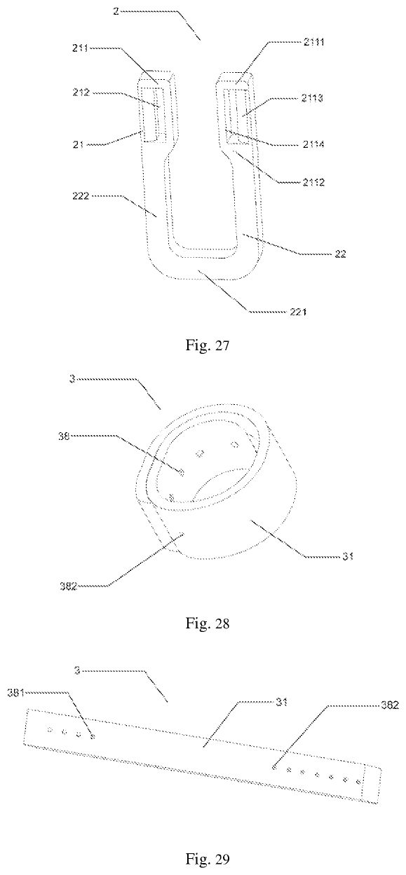

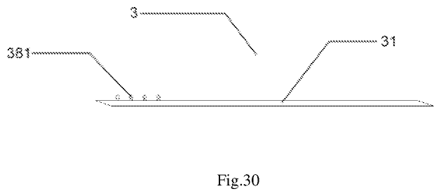

FIG. 25, FIG. 26, FIG. 27, FIG. 28, FIG. 29, and FIG. 30 show a third preferred embodiment of the present invention. The overall structure of the internal nasal dilator 1 in this embodiment is similar to that in the first embodiment. Special parts of this embodiment are as follows: First, the fixing clip 21 and the intranasal stent 3 are slightly different from the corresponding structures in the first embodiment. Second, a fixing manner is different. The intranasal stent 3 in this embodiment is first self-fixed by an end-to-end overlapping region to form an annular structure and is subsequently fixed on the fixing clip 21, and the fixing position of the intranasal stent 3 on the fixing clip 21 is adjustable. The fixing position of the intranasal stent 3 in the first embodiment is not adjustable. Third, a size adjustment manner is different. In this embodiment, the size of the intranasal stent 3 is adjusted by adjusting a relative position of self-fixing of the intranasal stent 3. In the first embodiment, the size of the intranasal stent 3 is adjusted by adjusting the fixing position of the intranasal stent 3 on the fixing clip 21.

As shown in FIG. 27, the structure of the fixing clip 21 is substantially the same as that of the fixing clip in the first embodiment in FIG. 8, and a difference lies in that the fixing clip 21 in this embodiment does not include the clip opening 213. Moreover, the inner wall 2113 in this embodiment uses the optimized structure in FIG. 12. The width on a side of the inner wall 2113 that is close to the fixing hole 212 decreases, thereby enhancing the clamping and fixation of the intranasal stent 3 by the fixing clip 21. In other similar embodiments, a sidewall of the fixing hole 212 may not have a decreasing width on the side close to the fixing hole 212.

As shown in FIG. 28, FIG. 29, and FIG. 30, the intranasal stent 3 includes a stent body 31 and a snap fastener 38. The snap fastener 38 includes a group of snap fastener plugs 381 and a group of snap fastener holes 382. Two ends of the ribbon-shaped stent body 31 are wedge-shaped structures in FIG. 30. A plurality of snap fastener holes 382 that penetrate the stent body 31 and are arranged at equal intervals are arranged on a midline of the stent body 31 that is close to one end thereof; the number of the snap fastener holes 382 may be increased or reduced according to an actual requirement, and is preferably 7; a plurality of snap fastener plugs 381 that are arranged at equal intervals and have spacings equal to those of the snap fastener holes 382 are integrally arranged on the midline of the stent body 31 that is close to the other end thereof, and the snap fastener plugs 381 protrude from the surface of the stent body 31; and the number of the snap fastener plugs 381 may be increased or reduced according to an actual requirement, and is preferably 4. The snap fastener plugs 381 can be inserted and fixed in the snap fastener holes 382, thereby closing the snap fastener 38. To reliably fix the intranasal stent 3, the length of the fixing hole 212 is the same as the width of the expanded stent body 31, the width of the fixing hole 212 is slightly greater than the thickness of the stent body 31 and is less than twice the thickness of the stent body 31, thereby ensuring that the fixing hole 212 can clamp, press, and fix the overlapping position of the intranasal stent 3.

As shown in FIG. 25, FIG. 26, FIG. 27, FIG. 28, FIG. 29, and FIG. 30, an assembly method of the internal nasal dilator 1 in this embodiment is as follows: An end of the expanded intranasal stent 3 is inserted into and passed through the fixing hole 212. Subsequently, the intranasal stent 3 is coiled to form an annular structure to make the intranasal stent 3 overlap end-to-end, after the perimeter of a suitable annular structure is chosen, and the snap fastener 38 is closed, that is, four snap fastener plugs 381 at corresponding positions are inserted and fixed in four snap fastener holes 382, thereby fixing the intranasal stent 3 as annular structure using the snap fastener 38 and lock the size of the intranasal stent 3. In this case, surfaces of larger sides of wedge-shaped structures at two ends of the stent body 31 are close to the stent body 31, to make surfaces of an outer side and an inner sides of the annular intranasal stent 3 remain smooth, thereby smoothly completing subsequent adjustment. Next, the position of the annular intranasal stent 3 in the fixing hole 212 is adjusted, so that the fixing hole 212 surrounds the end-to-end overlapping region of the intranasal stent 3. Because the width of the fixing hole 212 is less than the thickness of the overlapping region of the intranasal stent 3, the fixing hole 212 clamps the intranasal stent 3, and the frictional force between the two fixes intranasal stent 3 on the fixing clip 21. Finally, the user can adjust the size of the intranasal stent 3 by selecting different combinations of the snap fastener plugs 381 and the snap fastener holes 382.

As a further optimization, to enhance the fixation of the intranasal stent 3 by the fixing clip 21, the clip teeth may alternatively be arranged on the inner side of the annular intranasal stent 3.

As a further optimization, to enhance the fixation of the intranasal stent 3 by the fixing clip 21, the serrated or undulating structure may alternatively be arranged on the upper side and/or the lower side of the annular intranasal stent 3.

In addition, various optimizations made in the first embodiment and the second embodiment as well as the wearing method in the first embodiment are also applicable to this embodiment.

Compared with the first embodiment, the advantages of this embodiment are as follows: The clip opening 213 does not need to be arranged for the nose clip 2, and therefore the intranasal stent 3 does not easily fall off from the nose clip 2. The connection position between the intranasal stent 3 and the fixing clip 21 has a simpler structure, so that a respiratory resistance is smaller. The weaknesses of this embodiment are as follows: The structure of the intranasal stent 3 is more complex, and manufacturing difficulty is higher. The snap fastener hole 382 reduces the strength and the support force of the intranasal stent 3 at this position.

Finally, it should be noted that the foregoing embodiments are merely intended for describing the technical solutions of the present invention rather than limiting the present invention. Although the present invention is described in detail with reference to the embodiments, persons of ordinary skill in the art should understand that they may still make modifications or equivalent replacements to the technical features of the present invention without departing from the spirit and scope of the technical solutions of the technical solutions of the present invention. These modifications or equivalent replacements shall all fall within the scope of the claims of the present invention.

1. An internal nasal dilator, wherein the internal nasal dilator comprises one nose clip and two intranasal stents;
the nose clip comprises one substantially U-shaped or C-shaped nose clip body and two fixing clips that are symmetrically arranged and are integrally connected to distal ends of two branches of the nose clip body; and
each of the intranasal stents self-expands into a flat ribbon-shaped structure, is detachably connected and fixed to each of the fixing clips of the nose clip, and coils to form an annular structure with an adjustable perimeter.

2. The internal nasal dilator according to claim 1, wherein an axis of the annular structure is substantially parallel to an axis of a nasal passage in which the annular structure is located.

3. The internal nasal dilator according to claim 1, wherein the fixing clip of the nose clip comprises an annular clip frame, a vertically penetrating fixing hole is provided in the clip frame, and a clip opening is provided on an outer side of the fixing hole;
the intranasal stent comprises a stent body and a stent tail;
the stent tail is integrally connected to an end of the stent body, the stent tail comprises an annular frame, the frame is provided with an insertion hole penetrating the frame, and the frame of the stent tail surrounds and is fixed to the clip frame; and
a portion of the stent body is fixed in the fixing hole and cooperates with the fixing clip to adjust and fix the perimeter of the annular structure.

4. The internal nasal dilator according to claim 1, wherein the fixing clip of the nose clip comprises an annular clip frame, a vertically penetrating fixing hole is provided in the clip frame, the fixing hole comprises an inner hole and an outer hole, and a partition is provided between the inner hole and the outer hole;
the intranasal stent comprises a stent body and a stent tail;
the stent tail is integrally connected to an end of the stent body, a tail end is provided at an end of the stent tail that is away from the stent body, a lateral perimeter of the tail end is greater than a lateral perimeter of the stent tail, a protruding tail tooth is provided at an end of the stent tail that is close to the stent body, the stent tail is inserted into the outer hole, and the tail end and the tail tooth are exposed from two ends of the outer hole to fix the intranasal stent and the nose clip to each other; and
a portion of the stent body is fixed in the inner hole and cooperates with the fixing clip to adjust and fix the perimeter of the annular structure.

5. The internal nasal dilator according to claim 3, wherein a plurality of clip teeth arranged at fixed intervals are further provided at the stent body of the intranasal stent and cooperate with the fixing clip to adjust and fix the perimeter of the annular structure.

6. The internal nasal dilator according to claim 3, wherein a serrated or undulating structure is arranged on an upper side and/or a lower side of the stent body of the intranasal stent and cooperates with the fixing clip to adjust and fix the perimeter of the annular structure.

7. The internal nasal dilator according to claim 1, wherein the fixing clip of the nose clip comprises an annular clip frame, and a vertically penetrating fixing hole is provided in the clip frame;
the intranasal stent comprises a stent body; and
self-fixing devices with an adjustable fixing position extend outward from two ends of the stent body and form the annular structure after coiled overlapping fixation.

8. The internal nasal dilator according to claim 1, wherein the nose clip and the intranasal stent are made of different materials, the nose clip is made of a rigid elastic material, and the intranasal stent is made of a soft elastic material.

9. The internal nasal dilator according to claim 1, wherein at least one ribbon-shaped support spoke with two ends integrally connected to an inner wall of an intranasal stent is arranged in the intranasal stent.

10. The internal nasal dilator according to claim 1, wherein a structure used for accommodating a compound is arranged on an inner side of the annular structure of each intranasal stent, and is capable of releasing the compound into the nasal passage or absorbing and filtering specific substances from airflow in the nasal passage.

11. The internal nasal dilator according to claim 4, wherein a plurality of clip teeth arranged at fixed intervals are further provided at the stent body of the intranasal stent and cooperate with the fixing clip to adjust and fix the perimeter of the annular structure.

12. The internal nasal dilator according to claim 4, wherein a serrated or undulating structure is arranged on an upper side and/or a lower side of the stent body of the intranasal stent and cooperates with the fixing clip to adjust and fix the perimeter of the annular structure.
